(19) United States
(12) Patent Application Publication
(10) Pub. No.: US 2016/0022977 A1
(43) Pub. Date: Jan. 28, 2016

(54) METHOD FOR MANUFACTURING A CAP

(71) Applicants: TERUMO KABUSHIKI KAISHA, Tokyo (JP); CORONA GIKEN INDUSTRIES, Matsudo-City (JP)

(72) Inventors: Yasuhiro UEDA, Kofu-shi (JP); Masaki MATSUZAWA, Fujinomiya-shi (JP); Takeshi TOYAMA, Nakakoma-gun (JP); Toshinori TSUJI, Matsudo-shi (JP)

(73) Assignees: TERUMO KABUSHIKI KAISHA, Tokyo (JP); CORONA GIKEN INDUSTRIES, Matsudo-City (JP)

(21) Appl. No.: 14/872,408

(22) Filed: Oct. 1, 2015

Related U.S. Application Data
(63) Continuation of application No. PCT/JP2013/002246, filed on Apr. 1, 2013.

(51) Int. Cl. A61M 39/10; B29C 45/00; A61M 39/24

(52) U.S. Cl. CPC A61M 39/105; A61M 39/24; B29C 45/0046; A61M 2039/2426; A61M 2207/00; B29L 2031/753

(57) ABSTRACT

A method of manufacturing a cap including a top face ring having an opening on the center thereof and a peripheral wall extending from the outer peripheral edge of the top face ring and directly or indirectly attached to a housing . When the cap is injection molded, a molten resin is injected from a center gate of a cavity of a circular plate located on the opening of the top face ring, allowed to radially flow to fill a cavity of the top face ring, and allowed to further flow to fill a cavity of the peripheral wall to form a molded body.

### CROSS-REFERENCE TO RELATED APPLICATION

This application is a continuation of International Application No. PCT/JP2013/002246 filed on Apr. 1, 2013, the entire content of which is incorporated herein by reference.

### TECHNICAL FIELD

The disclosure herein relates to a method for manufacturing a cap used for attaching a valve body to a housing having a flow path through which liquid passes and a connection hole communicating with the flow path, the valve body blocking the connection hole. In particular, the disclosure aims to avoid the formation of a weld line on a top face ring of the cap and thereby improve the strength of the cap.

### BACKGROUND DISCUSSION

Conventionally, there has been known, as a connector provided with a cap, a medical mixed injection port which is attached to a medical device performing liquid feeding for mixing and injecting liquid from the outside of a liquid feeding path or, on the contrary, extracting liquid from the inside of the liquid feeding path, for example, as described in JP 3389983 B2. As illustrated in FIGS. 1(a) to 1(c), the medical mixed injection port described in JP 3389983 B2 is provided with a housing 103 which has a flow path 101 and a connection hole 102 communicating with the flow path 101, a valve body 104 which blocks the connection hole 102, and a cap 105 for attaching the valve body 104 to the housing 103. In the housing 103, a mount 106 is formed around the connection hole 102. The valve body 104 can be attached to the housing 103 by placing an outer peripheral edge 104a of the valve body 104 on the mount 106, putting the cap 105 thereover from the upper side, and engaging a locking hole 105c of a locking piece 105b which extends downward from a peripheral wall 105a of the cap 105 with an engagement projection 103a which is formed on the outer side face of the housing 103.

Then, a male connector (not illustrated) is inserted into a fitting hole 107 which is defined by a top face ring 105d of the cap 105. Accordingly, a slit 108 of the valve body 104 is pushed open by the tip of the male connector, which enables the flow path 101 inside the housing 103 and a flow path inside the male connector to communicate with each other. At this point, the male connector can be locked to the cap 105 by fitting the male connector with the inner peripheral edge of the fitting hole 107. When the male connector is pulled out, the valve body 104 can be restored to its original shape to block the connection hole 102.

Conventionally, when a cap having such a structure is manufactured by injection molding resin, there has typically been used a method in which a gate is formed at a position corresponding to the lateral side of the cap, a molten resin is injected from the gate, the molten resin is allowed to flow to a part of a cavity of the cap, the part being located opposite to the gate, and the molten resin flows are allowed to join together in this part to form a molded body.

However, when the gate is formed on the lateral side to form a molded body in this manner, a weld line which extends in a radial direction is formed on the top face ring of the cap due to the joining of the flows of the molten resin. Thus, when an external force is applied to the lateral side of a male connector connected to the connector, the top face ring of the cap may be broken from the weld line which is a fragile site and the liquid-tightness of the connector may be disadvantageously lost depending on the size or the direction of the external force, the material of the male connector, or the material of the cap.

### SUMMARY

The disclosure herein provides a cap manufacturing method that makes it possible to avoid the formation of a weld line on a top face ring of a cap and thereby improve the strength of the cap.

A cap manufacturing method of the disclosure herein is directed to a method for manufacturing a cap used for attaching a valve body to a housing, the cap having a flow path through which liquid passes and a connection hole communicating with the flow path, the valve body blocking the connection hole. In the cap manufacturing method, the cap includes a top face ring having an opening on the center thereof and a peripheral wall hung from the outer peripheral edge of the top face ring and directly or indirectly attached to the housing. When the cap is injection molded, a molten resin is injected from a center gate of a cavity of a circular plate located on the opening of the top face ring, the molten resin is allowed to radially flow to fill a cavity of the top face ring, and the molten resin is allowed to further flow to fill a cavity of the peripheral wall to form a molded body.

Here, “cavity” indicates a gap that is formed by a mold used in the injection molding and filled with the molten resin.

In the cap manufacturing method of the disclosure herein, the circular plate is preferably punched and removed with the molded body held by a mold.

In the cap manufacturing method of the disclosure herein, an external thread is preferably formed on the outer peripheral face of the peripheral wall.

In an exemplary embodiment of the disclosure, a molten resin is injected from the center gate of the cavity of the circular plate which is located on the opening of the top face ring and allowed to radially flow to fill the cavity of the top face ring. Thus, it is possible to avoid flows of the molten resin from joining together inside the cavity of the top face ring. As a result, it is possible to reliably avoid the formation of a weld line on the top face ring and thereby significantly improve the strength of the cap.

### BRIEF DESCRIPTION OF THE DRAWINGS

FIGS. 1(a) to 1(c) are diagrams illustrating a conventional connector, specifically, FIG. 1(a) is a plan view, FIG. 1(b) is a partial sectional view taken along line A-A of FIG. 1(a), and FIG. 1(c) is a sectional view taken along line B-B of FIG. 1(a).

FIG. 2 is a longitudinal sectional view illustrating a cap manufactured by a cap manufacturing method according to an embodiment of the present invention in a state attached to a housing.

FIG. 3 is a plan view of the cap manufactured by the cap manufacturing method according to the embodiment of the present invention.

FIG. 4 is a sectional view taken along line C-C of FIG. 3.

FIG. 5 is a longitudinal sectional view illustrating a metal mold used in the cap manufacturing method according to the embodiment of the present invention.

FIG. 6 is a longitudinal sectional view illustrating a procedure for punching and removing a circular plate and an annular thin section of the cap using the metal mold illustrated in FIG. 5

### DETAILED DESCRIPTION

Hereinbelow, a cap manufacturing method according to an exemplary embodiment of the disclosure will be described as an example with reference to the drawings.

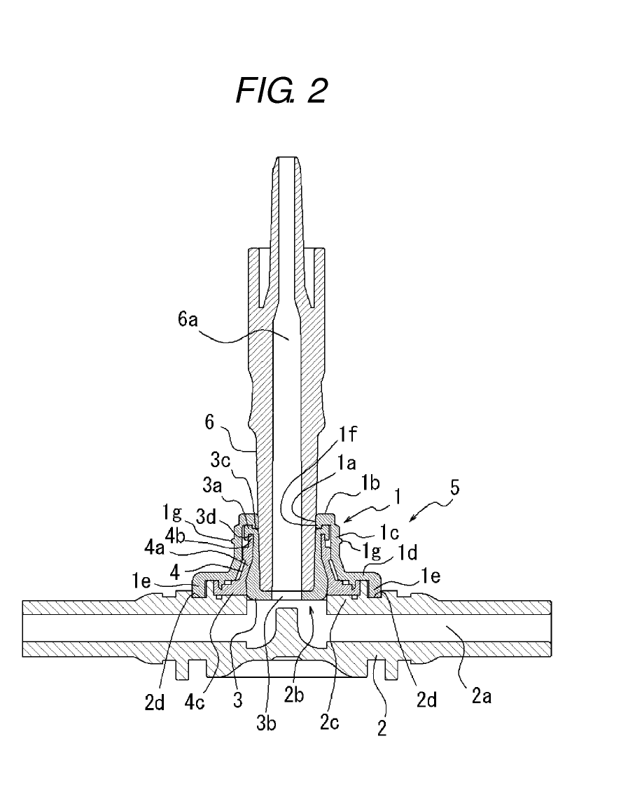

First, the configuration and the form of use of a cap manufactured by the cap manufacturing method according to the disclosure will be described with reference to FIG. 2. A cap 1 is used for attaching a valve body 3 to a housing 2. The cap 1 is provided with a top face ring 1b which has an opening 1a at the center thereof, a peripheral wall 1c extending from the outer peripheral edge of the top face ring 1b, an outward-facing flange 1d which is formed continuously with the lower end of the peripheral wall 1c, and a pair of positioning pieces 1e which face each other across the axis of the cap 1 on the bottom face of the outer peripheral edge of the outward-facing flange 1d.

The valve body 3 has a slit 3b on a central part thereof which is surrounded by an outer peripheral edge 3a and has a top face side annular groove 3c on the upper face of the outer peripheral edge 3a. The top face side annular groove 3c is liquid-tightly fittable with an annular projection 1f which is formed on the bottom face of the top face ring 1b of the cap 1. A bottom face side annular groove 3d is formed on the bottom face of the outer peripheral edge 3a of the valve body 3. An annular projection 4b which is formed on the upper end of a barrel 4a of an insertion body 4 is liquid-tightly fittable with the bottom face side annular groove 3d. An annular bottom face 4c which extends outward is integrally formed with the outer peripheral edge on the lower end of the barrel 4a of the insertion body 4.

A flow path 2a through which liquid passes and a connection hole 2b which communicates with the flow path 2a are formed in the housing 2. A mount 2c is formed around the connection hole 2b. A pair of positioning recesses 2d which face each other across the center of the connection hole 2b are formed on the outer peripheral edge of the mount 2c.

Thus, the outer peripheral edge 3a of the valve body 3 can be attached to the housing 2 by fitting the top face side annular groove 3c of the outer peripheral edge 3a of the valve body 3 with the annular projection 1f of the top face ring 1b of the cap 1, fitting the annular projection 4b of the insertion body 4 with the bottom face side annular groove 3d of the outer peripheral edge 3a of the valve body 3, inserting the pair of positioning pieces 1e of the cap 1 into the pair of positioning recesses 2d of the housing 2, and then fusion-bonding the outer peripheral edge of the outward-facing flange 1d of the cap 1 to the outer peripheral edge of the mount 2c of the housing 2. In this manner, the connector 5 is formed. With this configuration, the connection hole 2b of the housing 2 is blocked by the valve body 3.

Then, as illustrated in FIG. 2, a male connector 6 having a preferred tip outer diameter of 4.0 mm which is typically used in an infusion set is inserted into the opening 1a on the top face ring 1b of the cap 1, and the outer peripheral face of the male connector 6 is fitted with the inner peripheral edge of the opening 1a. Accordingly, the slit 3b of the valve body 3 is pushed open by the male connector 6, and a connection state between the male connector 6 and the connector 5 in which the flow path 2a of the housing 2 and a flow path 6a of the male connector 6 communicate with each other is maintained.

An external thread 1g is formed on the outer peripheral face of the peripheral wall 1c of the cap 1 for connecting a luer lock type male connector (not illustrated) having an internal thread. In the exemplary embodiment, the external thread 1g is a double-start thread which can be used with a standard luer lock type male connector.

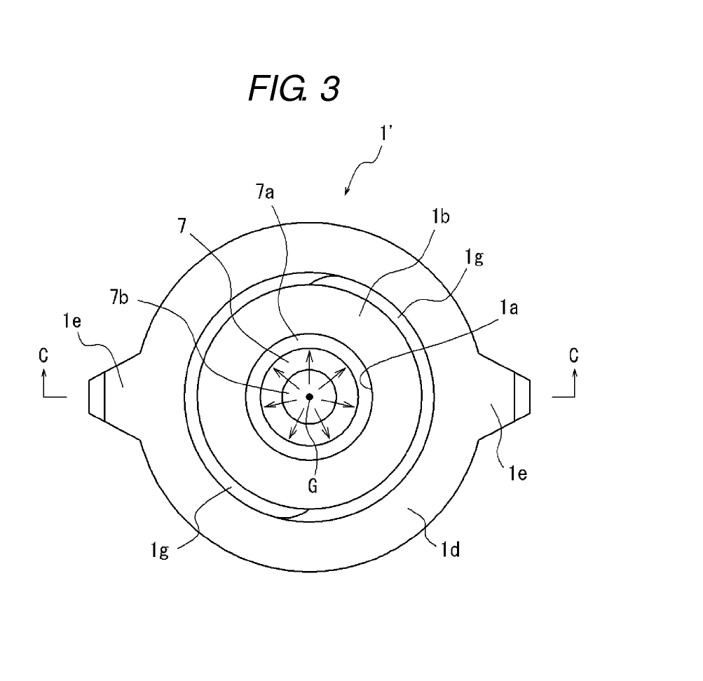

A method for manufacturing the cap having the configuration and the form of use as described above according to the exemplary embodiment will be described below with reference to FIGS. 3 to 6.

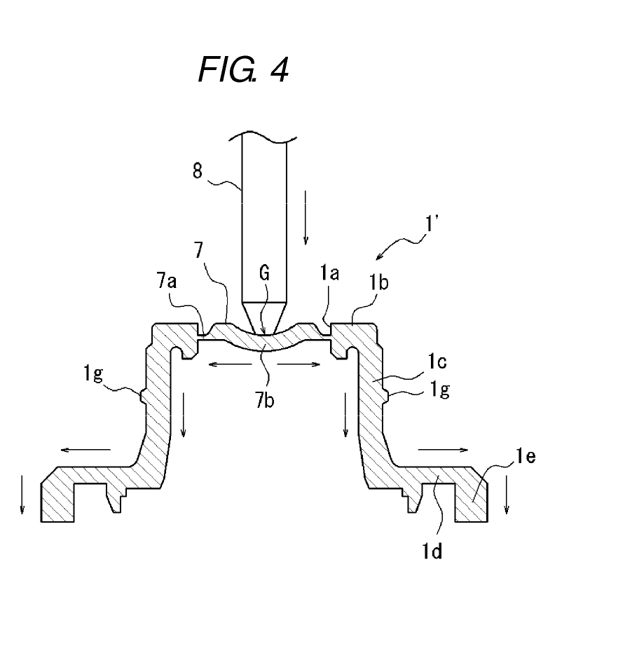

FIG. 3 is a plan view of an intermediate product (molded body) of the cap. FIG. 4 is a sectional view taken along line C-C of FIG. 3. The intermediate product 1′ has the same configuration as the cap 1 described above with reference to FIG. 2 except that the intermediate product 1′ includes a circular plate 7 which is arranged on the opening 1a of the top face ring 1b and an annular thin section 7a which is formed on the peripheral edge of the circular plate 7. As illustrated in FIGS. 3 and 4, the circular plate 7 has a curved section 7b which is curved downward in a dome-like shape. The peripheral edge of the circular plate 7 is integrally connected to the inner peripheral face of the opening 1a of the top face ring 1b through the annular thin section 7a. As illustrated in FIG. 4, a sprue 8 is connected to a center gate G which is located on the center of the circular plate 7.

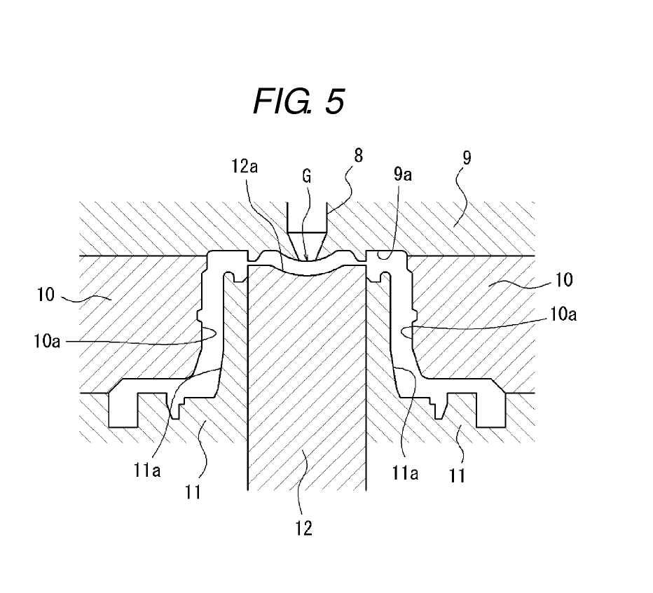

FIG. 5 illustrates a metal mold for molding the intermediate product of the cap. FIG. 5 illustrates a first upper mold 9, a second upper mold 10, a first lower mold 11, and a second lower mold 12. The first upper mold 9 includes the center gate G. The first upper mold 9 is provided with a molding surface 9a which forms the circular plate, the annular thin section, and the upper face of the top face ring. The second upper mold 10 is provided with a molding surface 10a which forms the outer peripheral face of the peripheral wall. The first lower mold 11 is provided with a molding surface 11a which forms the inner peripheral face of the peripheral wall. The second lower mold 12 is provided with a molding surface 12a which forms the bottom faces of the circular plate and the annular thin section.

When a molten resin is injection molded using the above metal mold, the resin flows in from the sprue 8, flows through a cavity of the circular plate in radial directions indicated by the arrows in FIG. 3 from the center gate G of the cavity, and flows through a cavity of the annular thin section 7a on the peripheral edge of the circular plate 7 to fill a cavity of the top face ring 1b. Then, the resin flows in directions indicated by the arrows in FIG. 4 to sequentially fill a cavity of the peripheral wall 1c, a cavity of the outward-facing flange 1d, and a cavity of each of the positioning pieces 1e. Thus, flows of the resin do not join together in the top face ring 1b. Therefore, it is possible to reliably avoid the generation of a weld line on the top face ring 1b. In the exemplary embodiment, it is possible to minimize the joining of flows of the resin also in the peripheral wall 1c and the outward-facing flange 1d to thereby minimize the generation of a weld line on the peripheral wall 1c and the outward-facing flange 1d.

Examples of the material of the molten resin include a polyolefin such as polyethylene, polypropylene, and an ethylene-propylene copolymer; an ethylene-vinyl acetate copolymer (EVA); polyvinyl chloride; polyvinylidene chloride; polystyrene; polyamide; polyimide; polyamide-imide; polycarbonate; poly(4-methyl-1-pentene); ionomer; an acrylic resin; polymethyl methacrylate; an acrylonitrile-butadiene-styrene copolymer (ABS resin); an acrylonitrile-styrene copolymer (AS resin); a butadiene-styrene copolymer; polyester such as polyethylene terephthalate (PET), polybutylene terephthalate (PBT), and polycyclohexane terephthalate (PCT); polyether; polyether ketone (PEK); polyether ether ketone (PEEK); polyether imide; polyacetal (POM); polyphenylene oxide; modified polyphenylene oxide; polysulfone; polyether sulfone; polyphenylene sulfide; polyarylate; aromatic polyester (a liquid crystal polymer); and polytetrafluoroethylene, polyvinylidene fluoride and other fluororesins. A blend or a polymer alloy containing one or more kinds of the above materials may also be used.

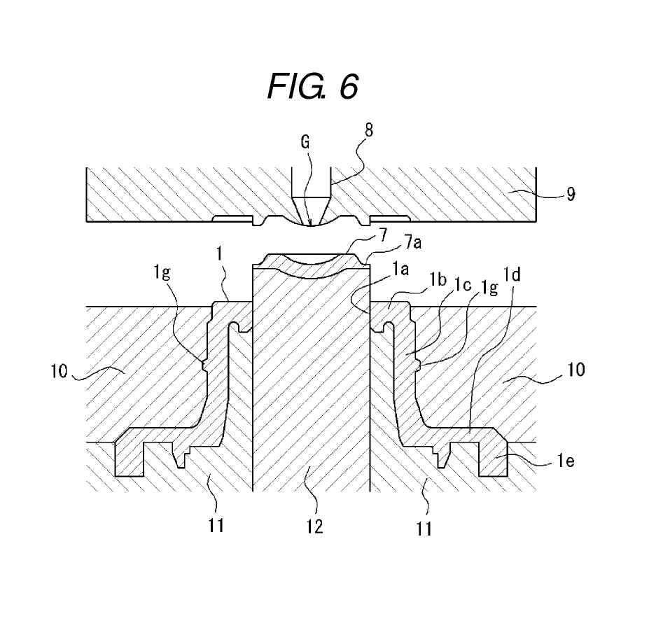

The molded body obtained in this manner is cooled and solidified inside the metal mold. Then, as illustrated in FIG. 6, the circular plate 7 is punched and removed together with the annular thin section 7a on the peripheral edge of the circular plate 7. Specifically, after the molded body is cooled and solidified, the second upper mold 10, the first lower mold 11, and the second lower mold 12 are moved downward with respect to the first upper mold 9 which serves as a fixed mold. Then, the second lower mold 12 is moved upward with respect to the second upper mold 10 and the first lower mold 11 to punch and remove the circular plate 7 and the annular thin section 7a. Punching the circular plate 7 and the annular thin section 7a with the molded body held by the molds in this manner enables unevenness in quality caused by deviation of a cut position to be reliably reduced compared to a case in which a molded body is taken out of a mold and cut with a cutting tool or laser.

After the punching and removal, the second upper mold 10 can be divided and moved to both lateral sides of the cap 1 to detach and take out the cap 1 therefrom.

The cap manufacturing method according to the exemplary embodiment described above makes it possible to reliably avoid the formation of a weld line on the top face ring of the cap and thereby significantly improve the strength of the cap.

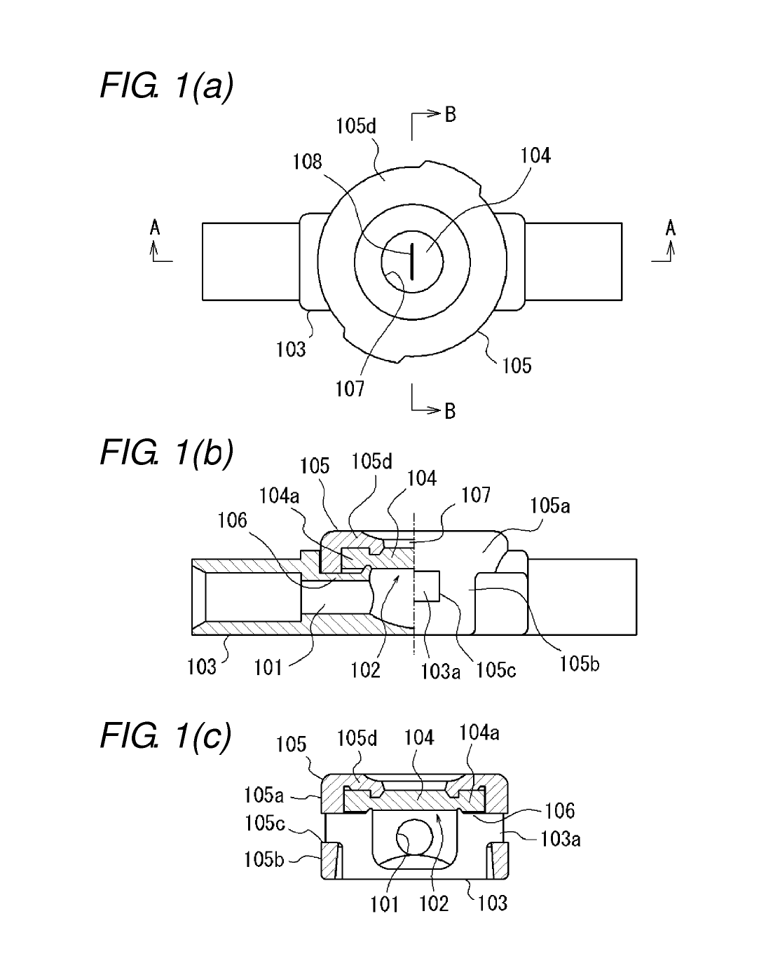

In the above, merely an exemplary embodiment of the disclosure herein has been described. Thus, various modifications may be added within the scope of the claims. For example, in the above description, the metal mold used in the injection molding of the cap includes the first upper mold, the second upper mold, the first lower mold, and the second lower mold. However, the metal mold is not necessarily required to have such a configuration, and the configuration of the metal mold may be appropriately modified taking into consideration, for example, the shape of the cap and the manufacturing efficiency. In the above description, the cap manufactured in the exemplary embodiment has the form in which the outward-facing flange is formed continuously with the lower end of the peripheral wall and the peripheral wall is attached to the housing through the outward-facing flange. However, the form of the cap is not limited to such a form. In the above description, the external thread is formed on the outer peripheral face of the peripheral wall of the cap for connecting a luer lock type male connector. However, such an external thread is not necessarily required and may be omitted. In the above description, the cap has the form used together with the insertion body. However, the cap is not limited to such a form. It is needless to say that, for example, the cap manufacturing method according to the disclosure herein may also be applied to the conventional cap illustrated in FIGS. 1(a)-1(c).

As an example, a polypropylene having a melt flow rate of 6.5 g/10 min was supplied to an injection molding machine (NPX7-1F manufactured by NISSEI PLASTIC INDUSTRIAL CO., LTD.) and injection molding was performed using the metal mold illustrated in FIG. 5 under the molding conditions of a cylinder temperature of 230° C., a mold temperature of 60° C., and a molding pressure of 700 kg/cm². The circular plate 7 and the annular thin section 7a were punched and removed from the molded intermediate product by the procedure illustrated in FIG. 6 to manufacture the cap 1. Then, 100 samples of the connector 5 illustrated in FIG. 2 were manufactured using the cap 1. The male connector 6 (a polycarbonate male connector manufactured by TERUMO CORPORATION) was inserted into each of the samples, and an external force of 50 N and 2 seconds was repeatedly applied 150 times to the rear end (the upper end in FIG. 2) of the male connector 6 at intervals of 90 seconds to thereby examine whether a crack is generated on the top face ring of the connector 5. As the result, no crack was confirmed in all of the 100 samples.

On the other hand, as a comparative example, a cap was manufactured by injection molding under the same conditions as the above molding conditions excepting that no circular plate and no annular thin section was provided and a molten resin was injected from one of the pair of positioning pieces of the cap. Then, in the same manner as in the above example, 100 samples of the connector 5 illustrated in FIG. 2 were manufactured using the cap to examine the presence/absence of a crack. As the result, the generation of a crack was confirmed in six of the 100 samples.

Thus, it has been confirmed from the above results that the cap manufacturing method according to the disclosure herein enables the strength of the cap to be significantly improved.

The detailed description above describes a method of manufacturing a cap. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A method for manufacturing a cap used for attaching a valve body to a housing having a flow path through which liquid passes and a connection hole communicating with the flow path, the valve body configured for blocking the connection hole, wherein the cap includes a top face ring having an opening on a center thereof and a peripheral wall extending from an outer peripheral edge of the top face ring and directly or indirectly attached to the housing, the method comprising:
injection molding the cap, said injection molding step including:
injecting a molten resin from a center gate of a cavity of a circular plate located on the opening of the top face ring;
radially flowing the molten resin to fill a cavity of the top face ring; and
radially flowing the molten resin to further fill a cavity of the peripheral wall to form a molded body.

2. The method for manufacturing a cap according to claim 1, further comprising punching the circular plate and removing the circular plate with the molded body held by a mold.

3. The method for manufacturing a cap according to claim 1, further comprising forming an external thread on an outer peripheral face of the peripheral wall.

4. A method for manufacturing a cap used for attaching a valve body to a housing having a flow path through which liquid passes and a connection hole communicating with the flow path, the valve body configured for blocking the connection hole, wherein the cap includes a top face ring having an opening at a center thereof, a peripheral wall extending from an outer peripheral edge of the top face ring and directly or indirectly attached to the housing, an outward-facing flange, and a pair of positioning pieces, the method comprising:
providing a mold having a first upper mold, a second upper mold, a first lower mold and a second lower mold, the first upper mold including a center gate, a molding surface which forms a circular plate, an annular thin section and an upper face of the top face ring;
injecting a molten resin from the center gate through of a cavity of the circular plate located on the opening of the top face ring;
radially flowing the molten resin through a cavity of the annular thin section to fill a cavity of the top face ring; and
flowing the molten resin to further fill a cavity of the peripheral wall to form a molded body.

5. The method for manufacturing a cap according to claim 4, further comprising flowing the molten resin to further fill a cavity of an outward-facing flange.

6. The method for manufacturing a cap according to claim 5, further comprising flowing the molten resin to further fill a cavity of each of the positioning pieces.

7. The method for manufacturing a cap according to claim 6, further comprising cooling and solidifying the molded body in the mold.

8. The method for manufacturing a cap according to claim 7, further comprising moving the second upper mold, the first lower mold and the second lower mold downward with respect to the first upper mold.

9. The method for manufacturing a cap according to claim 8, further comprising moving the second lower mold upward with respect to the second upper mold and the first lower mold, thereby punching the circular plate and annular thin section.

10. The method for manufacturing a cap according to claim 4, further comprising punching the circular plate and removing the circular plate with the molded body held by a mold.

11. The method for manufacturing a cap according to claim 4, further comprising forming an external thread on an outer peripheral face of the peripheral wall.
